SEQUENCE LISTING (27 sequences)

SEQ ID NO: 1 - DNA, 40 bp, synthetic construct
  source 1..40: mol_type = other DNA
  misc_feature 40: note = 3' biotin
ctaagtatcatatagttttttttttttttttttttttttt

SEQ ID NO: 2 - DNA, 40 bp, synthetic construct
  source 1..40: mol_type = other DNA
  misc_feature 1: note = 5' biotin
tttttttttttttttttttttttttcgtgatgtaatctgc

SEQ ID NO: 3 - DNA, 35 bp, synthetic construct
  source 1..35: mol_type = other DNA
  misc_feature 1: note = 5' Amino Modifier C6
tttttttttttttttcagtatgaagtgaactagtc

SEQ ID NO: 4 - DNA, 35 bp, synthetic construct
  source 1..35: mol_type = other DNA
  misc_feature 35: note = 3' Amino Modifier
cattctagagattgtgactattttttttttttttt

SEQ ID NO: 5 - DNA, 55 bp, synthetic construct
  source 1..55: mol_type = other DNA
ctaagtatcatatagttttttttttttttttttttgactagttcacttcatactg

SEQ ID NO: 6 - DNA, 55 bp, synthetic construct
  source 1..55: mol_type = other DNA
tagtcacaatctctagaatgttttttttttttttttttttcgtgatgtaatctgc

SEQ ID NO: 7 - DNA, 34 bp, synthetic construct
  source 1..34: mol_type = other DNA
ctatatgatacttagtgtagcagattacatcacg

SEQ ID NO: 8 - DNA, 35 bp, synthetic construct
  source 1..35: mol_type = other DNA
ctatatgatacttagttgtagcagattacatcacg

SEQ ID NO: 9 - DNA, 36 bp, synthetic construct
  source 1..36: mol_type = other DNA
ctatatgatacttagttgtatgcagattacatcacg

SEQ ID NO: 10 - DNA, 37 bp, synthetic construct
  source 1..37: mol_type = other DNA
ctatatgatacttagttgtatggcagattacatcacg

SEQ ID NO: 11 - DNA, 21 bp, synthetic construct
  source 1..21: mol_type = other DNA
gtaatctgctacactaagtat

SEQ ID NO: 12 - DNA, 23 bp, synthetic construct
  source 1..23: mol_type = other DNA
gtaatctgctacaactaagtatc

SEQ ID NO: 13 - DNA, 24 bp, synthetic construct
  source 1..24: mol_type = other DNA
gtaatctgcatacaactaagtatc

SEQ ID NO: 14 - DNA, 25 bp, synthetic construct
  source 1..25: mol_type = other DNA
gtaatctgccatacaactaagtatc

SEQ ID NO: 15 - DNA, 46 bp, synthetic construct
  source 1..46: mol_type = other DNA
caataactactcaatctcgatacttagtgtagcagattaccaactc

SEQ ID NO: 16 - DNA, 47 bp, synthetic construct
  source 1..47: mol_type = other DNA
caataactactcaatctcgatacttagttgtagcagattaccaactc

SEQ ID NO: 17 - DNA, 48 bp, synthetic construct
  source 1..48: mol_type = other DNA
caataactactcaatctcgatacttagttgtatgcagattaccaactc

SEQ ID NO: 18 - DNA, 49 bp, synthetic construct
  source 1..49: mol_type = other DNA
caataactactcaatctcgatacttagttgtatggcagattaccaactc

SEQ ID NO: 19 - DNA, 21 bp, synthetic construct
  source 1..21: mol_type = other DNA
gttggtaatctgctacactaa

SEQ ID NO: 20 - DNA, 22 bp, synthetic construct
  source 1..22: mol_type = other DNA
gttggtaatctgctacaactaa

SEQ ID NO: 21 - DNA, 23 bp, synthetic construct
  source 1..23: mol_type = other DNA
gttggtaatctgcatacaactaa

SEQ ID NO: 22 - DNA, 24 bp, synthetic construct
  source 1..24: mol_type = other DNA
gttggtaatctgccatacaactaa

SEQ ID NO: 23 - DNA, 38 bp, synthetic construct
  source 1..38: mol_type = other DNA
  misc_feature 1: note = 5' Thiol Modifier C6 S-S
tttttttttttttttgagttggtaatctgctacactaa

SEQ ID NO: 24 - DNA, 39 bp, synthetic construct
  source 1..39: mol_type = other DNA
  misc_feature 1: note = 5' Thiol Modifier C6 S-S
tttttttttttttttgagttggtaatctgctacaactaa

SEQ ID NO: 25 - DNA, 40 bp, synthetic construct
  source 1..40: mol_type = other DNA
  misc_feature 1: note = 5' Thiol Modifier C6 S-S
tttttttttttttttgagttggtaatctgcatacaactaa

SEQ ID NO: 26 - DNA, 41 bp, synthetic construct
  source 1..41: mol_type = other DNA
  misc_feature 1: note = 5' Thiol Modifier C6 S-S
tttttttttttttttgagttggtaatctgccatacaactaa

SEQ ID NO: 27 - DNA, 33 bp, synthetic construct
  source 1..33: mol_type = other DNA
  misc_feature 33: note = 3' Amino Modifier
gagattgagtagttattgttttttttttttttt

(19) United States
(12) Patent Application Publication
(10) Pub. No.: US 2026/0056190 A1
(43) Pub. Date: Feb. 26, 2026

(54) Ultrasensitive Protein Detection using a Proximity Immunoassay with Nucleic Acid Target Amplification and Photonic Resonator Absorption Microscopy (PINATA)

(71) Applicant: THE BOARD OF TRUSTEES OF THE UNIVERSITY OF ILLINOIS, Urbana, IL (US)

(72) Inventors: Skye T. Shepherd, Urbana, IL (US); Brian T. Cunningham, Champaign, IL (US)

(21) Appl. No.: 19/280,685

(22) Filed: Jul. 25, 2025

Related U.S. Application Data
(60) Provisional application No. 63/676,207, filed on Jul. 26, 2024.

(51) Int. Cl. G01N 33/53; G01N 33/543; G01N 33/553; G01N 33/68

(52) U.S. Cl. CPC G01N 33/5308; G01N 33/54346; G01N 33/553; G01N 33/6869; G01N 2333/5412

(57) ABSTRACT

The current disclosure provides an improved sensitivity assay for detecting and quantifying proteins in a sample. The assay utilizes a bridge sequence, a reporter sequence, two proximity probes, a solution; one or more adapters each tethered to an antibody; and a gold nanoparticle (AuNP) probe. The disclosure also provides a system and method for use of the assay.

### CROSS-REFERENCE TO RELATED APPLICATIONS

This application claims priority to U.S. Provisional Patent Application No. 63/676,207, filed Jul. 26, 2024, which is incorporated herein by reference.

### REFERENCE TO A SEQUENCE LISTING

The instant application contains an electronic Sequence Listing that has been submitted electronically and is hereby incorporated-by-reference in its entirety. The sequence listing was created on Jul. 24, 2025, is named “24-0923-US_Sequence-Listing_FINAL.xml”, and is 28,827 bytes in size.

### BACKGROUND

Protein biomarkers can provide valuable insights into various disease states, particularly when considering posttranslational modifications, degradation, or various protein-protein interactions. Proteins have been identified as biomarkers for cancer, cardiovascular disease, sepsis, traumatic brain injury, neurological disease and more, allowing for early screening, predictive monitoring, and personalized therapeutics in many diseases. For instance, protein detection can provide a real-time profile of biomarkers when compared to genetic and transcriptome sequencing. Antigen and antibody detection are also effective at diagnosing and controlling infectious diseases, and are valuable for rapid viral testing, immunology, or vaccine development. Non-traditional affinity groups such as aptamers, peptides, single chain fragment variables, and nanobodies are affinity-based protein techniques that are powerful, flexible tools for molecular biology and biosensing. However, the sensitivity of protein detection has typically lagged behind nucleic acids, where powerful exponential amplification techniques like polymerase chain reaction (PCR), loop-mediated isothermal amplification (LAMP), and rolling circle amplification (RCA) allow detection in the attomloar to femtomolar range. In contrast, protein measurements using conventional immunoassays, such as enzyme-linked immunosorbent assays (ELISAs) show limits of detection (LOD) in the high picomolar range, with most commercial biological assays struggling to detect below 10 pg/mL. Greater sensitivity in protein testing leads to faster protein detection for point-of-care applications, earlier disease detection, smaller sample volumes, easier detection of certain targets in healthy individuals, more potential new protein biomarkers, and more.

Ultrasensitive protein detection includes digital detection of individual proteins to increase sensitivity, using assays such as single molecule arrays (SiMoA). The arrays have been used in digital assays using fluorescent individual compartmentalized beads to detect individual proteins, with detection limits in the high attomolar and low femtomolar range. However, SiMoAs have long assay times and require special equipment and trained personnel.

An alternative approach to increase sensitivity in protein detection is to utilize nucleic acid amplification techniques. For instance, immuno-PCR uses a sandwich assay approach, where a target protein is immobilized on a surface between two antibodies. The detection antibody is tagged with a DNA sequence that is amplified and detected using qPCR. Through the enzymatic exponential amplification, immuno-PCR can achieve greater sensitivity (LOD of ˜500 fg/mL) than a sandwich assay alone, although it still can require time-intensive protocols (4+ hours), extensive washing to ensure low backgrounds, and thermocycling.

Additional approaches include proximity ligation assays (PLAs) and proximity extension assays (PEAs). The proximity-based affinity assays involves two DNA-labeled antibodies or aptamers that bind to the same protein on different epitopes, thereby triggering ligation or extension. Nucleic acid amplification techniques are then applied to amplify the signal for bulk detection. The proximity assays utilize enzymatic nucleic acid amplification with antibody-oligonucleotide conjugates to increase signal detection and can be performed homogenously or on fixed cells. This allows for faster and more sensitive assays without requiring intensive washing. PLAs typically use DNA-labeled antibody proximity probes with free oligonucleotides that are joined by DNA ligase when brought into close range by a target. DNA polymerase is then added and the signal amplified and detected using qPCR or RCA. PLAs have been used with various methods to detect targets in the high femtomolar to nanomolar range, such as proteins, cytokines, exosomes, and viral antigens and have been adapted for the study of protein-protein interactions or post-translational protein modifications.

However, the utility of PLAs is limited by interference in biological samples. As a result, proximity extension assays are used for sensing technologies. Proximity assays utilize short complimentary regions that hybridize in the presence of a target protein followed by extension. The resulting DNA template can be detected by qPCR or by barcoding next-generation sequencing technologies.

PEA assays are standard proteomics tools and have a LOD of 10 fg/mL LOD to 20 fg/mL LOD). However, proximity assays, are enzyme dependent, require complicated storage, and present stability challenges. Enzymatic amplification utility is limited in point-of-care application because of the requirements for heating and/or thermocycling. Further, many proximity assay signal readouts rely on bulk fluorescence, which requires expensive lab equipment for high sensitivity detection.

Non-enzymatic proximity approaches have been explored, such as DNA entropy-driven cascade reactions. Proximity hybridization chain reaction (ProxHCR) was introduced as an alternative to enzymatic amplification, using proximity probes conjugated to antibodies to initiate hybridization chain reactions for target amplification. When brought together, the proximity probes with hairpin structures triggered isothermal (37° C.) linear amplification using HCR to increase the fluorescence signal generated. However, without exponential enzymatic amplification, the LOD was much higher (nanomolar) than PCR-based assays, even when using an expensive epifluorescence microscope. ProxHCR is typically used for fixed cells and studying protein:protein interactions, although electrochemiluminescence has been used with proxHCR as well. Another approach combined HCR with catalyzed hairpin assembly (CHA), and achieved a 0.73 pg/mL limit detection of prostate-specific antigen (PSA) through a 4-hour long procedure with an electrochemiluminescent approach. A thrombin aptamer-based proximity assay using fluorescent CHA demonstrated an 8.3 pg/mL limit of detection in about two hours. Electrochemical readouts have also been used with proximity assays for the detection of PSA in a ng/mL range. Nucleic acid toehold nanoswitches have been demonstrated with peptides for antibody detection with proximity assays. However, in general, proximity methods without enzymatic amplification have struggled to achieve rapid assay times or high sensitivity.

Thus, proximity methods without enzymatic amplification have struggled to achieve more rapid assay times or greater selectivity. As such, a need remains in the field for an alternative approach to enhance protein detection sensitivity.

### SUMMARY

The disclosure demonstrates an improved sensitivity assay, termed the Proximity Initiated Nucleic Acid Target Amplification (PINATA) assay for detecting and quantifying proteins in a sample. The disclosure also provides a system and method for use of the assay. The assay utilizes target recycling amplification process (TRAP) for the digital detection of proteins in tandem with photonic resonator absorption microscopy (PRAM) and utilizes toehold-mediated DNA strand displacement reactions. The disclosed assay, system, and methods provide enhanced sensitivity to detect and quantify nucleic acids in a biological sample and can be used in numerous settings including health status, early disease and medical condition diagnosis, identification of biomarkers, evaluation of therapeutic efficiency, and longitudinal monitoring of disease progression.

In one aspect of the disclosure is a system for quantifying proteins in a sample, comprising a bridge sequence, a reporter sequence, two proximity probes, a solution, one or more adapters each tethered to an antibody, a gold nanoparticle (AuNP) probe, a photonic crystal a sample, and an imaging platform, wherein the bridge sequence and reporter sequence are annealed to form a stable bridge-reporter duplex with a exposed nucleotide toehold region at each end of the bridge sequence; and wherein the proximity probes react with the toehold regions, further wherein simultaneous toehold mediated displacement occurs between the proximity probes and the bridge sequence in the presence of a target protein thereby releasing the reporter sequence, allowing the reporter sequence to activate a target recycling amplification process (TRAP) through strand displacement reactions thereby tethering an AuNP probe; wherein the imaging platform is configured to visualize and quantify the tethered AuNP probes using photonic resonator adsorption microscopy (PRAM).

In one aspect of the disclosure is a biologic assay comprising a bridge sequence, a reporter sequence, two proximity probes, a solution, one or more adapters each tethered to an antibody; and a sample; wherein the bridge sequence and reporter sequence are annealed to form a stable bridge-reporter duplex with an exposed toehold region at each end of the bridge sequence and the proximity probes bound to an antibody-linked adapter can bind a target antigen.

In one aspect of the disclosure is a method for detecting and/or quantifying proteins in a sample, comprising the steps of annealing a bridge sequence and a reporter sequence to form a stable bridge-reporter duplex with a six-nucleotide exposed toehold region at each end of the bridge sequence; simultaneous toehold displacement of one or more proximity probes and the bridge sequence in the presence of a sample containing a target protein thereby releasing a reporter sequence; activation of a target recycling amplification process (TRAP) through strand displacement reactions thereby tethering an AuNP probe; and visualizing and quantifying tethered AuNP using photonic resonator adsorption microscopy (PRAM).

### BRIEF DESCRIPTION OF THE DRAWINGS

FIGS. 1a-1c is a schematic of the proximity initiated nucleic acid target amplification (PINATA) assay. FIG. 1a is a schematic of a dual toehold design on an annealed bridge-reporter duplex has complementary regions to antibody-oligonucleotide proximity probes.

FIG. 1b is a schematic of when a target protein is present, the reporter is released through simultaneous toehold-mediated strand displacement reactions, converting the protein into a single-stranded DNA reporter sequence. FIG. 1c is a schematic of linear amplification of the reporter sequence occurs through toehold-mediated strand displacement reaction through TRAP. Photonic resonator adsorption microscopy is used to visualize and digitally count each bound AuNP.

FIGS. 2a-2c demonstrate initial biotin-streptavidin testing of system for feasibility and to thermodynamically tune reporter release. FIG. 2a is a 12% Native PAGE of proximity probes and target recycling amplification process for four different bridge-reporter lengths, reacted for 1 hour at room temperature. Blue arrow shows final triplex of a bound AuNP (capture-linker-NP probe). FIG. 2b is a schematic of DNA reporter release was tuned using biotinylated sequences with streptavidin to initially test proximity probes. BR5 is shown, with 5 nucleotides in region “c”. FIG. 2c is target recycling amplification test of annealed and purified BRs. 15% Native PAGE was run after reaction of capture, LP, NP probe, and BR for 4 hours at room temperature. Blue arrow indicates region of final bound triplex, with little obvious amplification even after 4 hours.

FIG. 3 demonstrates native PAGE testing of PINATA sequences for human IL-6 detection. Monoclonal and polyclonal antibody-oligonucleotide (AbO) conjugates with adapter sequences were compared for proximity probe use. All DNA sequences reacted at a concentration of 100 nM. The monoclonal and polyclonal AbOs (mAPP1, mAPP2, pAPP1, pAPP2) were added at a concentration of 20 nM and were preannealed to the two proximity probe sequences at a concentration of 100 nM. For a positive control, in Lane 2, a 1 nM concentration of human IL-6 was tested. In Lanes 3 and 5, a 10 nM concentration of IL-6 was tested. All sequences were reacted for two hours, then run on a 15% Native PAGE.

FIG. 4 is a processed PRAM images of counted AuNPs and standard curve for the target recycling amplification process (TRAP) of the reporter 5 sequence (R5). A negative control with no bridge-reporter duplex (blank) was compared to a 1 pM concentration of annealed and purified BR5. Two replicates were repeated for each concentration, and images were captured at 10, 20, and 30 minutes. The PRAM images are from 20 minutes. Standard error is shown (n=2). LOD threshold was calculated as three standard deviations above the background (3σ+blank)

FIG. 5 demonstrates an initial test of human IL6 on PRAM using PINATA BR5 probe set with monoclonal antibodies. Proximity probes were added to samples for 15 minutes, then TRAP sequences and AuNPs were added and imaged after 15 minutes.

FIGS. 6a-6b demonstrate the structure of a photonic crystal slab. FIG. 6a is the structure of a 1D grating photonic crystal slab, with low refractive index SiO2 and high refractive index TiO2 layers. FIG. 6b demonstrates Reflectivity of a photonic crystal surface in 2 mm depth of 1×TE buffer, tuned to highly reflect light at a wavelength of 622-625 nm.

FIGS. 7a-7b demonstrate NanoUrchins. FIG. 7a is a Transmission Electron Microscopy (TEM) images of spiky 80 nm gold NanoUrchins (AuNPs). FIG. 7b is an Absorption spectrum of the AuNPs, with a peak absorption of 625 nm.

FIG. 8 is a native PAGE validation of bridge-reporter annealing and purification of excess bridge for four different sets of bridge-reporter lengths. Lane 1: Bridge sequence (B). Lane 2: Reporter sequence (R). Lane 3: BR sequences annealed at a 1.2:1 stoichiometric ratio. Lane 3: BR sequences after excess bridge is purified.

FIG. 9 is a native PAGE of target recycling amplification process (TRAP) sequences. Lanes 1, 3, 5 and 7 are a negative control, with all TRAP sequences (capture, linker-protector (LP), and NP probe) added excluding reporter. Lanes 2, 4, 6, and 8 are a positive control and contain all TRAP sequences as well as one equivalent of the reporter sequence for reporters 4, 5, 6, and 7 to test recycling. The formation of the capture-linker-NP probe sequence indicates a final bound nanoparticle in the PRAM system.

FIG. 10 demonstrates Dynamic Light Scattering (DLS) measurements for hydrodynamic radius of the 80 nm gold NanoUrchins before and after conjugation to the thiol-terminated ssDNA probe sequences Pr5, Pr6, and Pr7. Error bars represent measurements of three sets of NPs, the mean radius of conjugated NPS is compared to the bare gold NPs by one-way ANOVA.

FIG. 11 is a NUPACK software prediction of structure at 25° C. for A1, P1. A2, P2, and B5 combination.

FIG. 12 demonstrates Native PAGE testing of PINATA sequences for human IL-6 detection using the purified BR5 sequence set. Monoclonal and polyclonal antibody-oligonucleotide (AbO) conjugates with adapter sequences were compared for proximity probe use. All DNA sequences reacted at a concentration of 100 nM. The monoclonal and polyclonal AbOs (mAbOP1, mAbOP2, pAbOP1, pAbOP2) were added at a concentration of 20 nM and were pre-annealed to the two proximity probe sequences at a concentration of 50 nM. Excess unconjugated proximity probe sequences were removed using magnetic beads. Immunoblot, lanes 1 and 2 show a positive and negative control with and without free reporter for target amplification. For a positive control, a 10 nM concentration of human IL-6 was tested in Lane 4 and 6, for monoclonal and polyclonal antibody oligonucleotide conjugates. Lanes 3 and 5 show a negative control without IL-6 target. All sequences were reacted for two hours at room temperature, then run on a 15% Native PAGE.

FIGS. 13a-13c demonstrate testing of human IL-6 using PINATA on PRAM. FIG. 13(a) is a control concentration of 0 pg/mL of IL-6. FIG. 13(b) shows detection using 10 pg/mL of IL-6, which can be detected in a thirty-minute reaction time. Counted images from PRAM are shown. FIG. 13(c) demonstrates counts wherein the left bar in each pair (c) at 30 min and 1 hour represents 0 pg/mL of IL-6 and the right bar in panel (c) represents 10 pg/mL of IL-6.

FIG. 14 demonstrates the nanoparticle count with increasing concentrations of IL-6. Approximately 1/100 reporters are removed by the target. Counts were collected after 30 minutes on PRAM.

### DETAILED DESCRIPTION

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

“Sample” as used herein refers to any type of sample, containing a nucleotide sequence and encompasses biological sample. “Biological sample” refers to a sample of body tissue, including but not limited to an organ punch or tissue biopsy, or fluid, including but not limited to blood, cerebrospinal fluid, plasma, or saliva from a warm-blooded animal such as a mammal, preferably a human, which is afflicted with, or has the potential to be afflicted with one or more diseases and/or disorders described herein. A biological sample can also refer to tissue or blood samples obtained from non-human mammals and other animals. In an alternative embodiment the sample is from a non-mammal host, which may contain the target nucleotide sequence.

In one aspect, the disclosure provides a system for quantifying proteins in a sample, comprising a bridge sequence; a reporter sequence; two proximity probes; a solution; one or more adapters each tethered to an antibody; a gold nanoparticle (AuNP) probe; a photonic crystal a sample; and an imaging platform, wherein the bridge sequence and reporter sequence are annealed to form a stable bridge-reporter duplex with a exposed nucleotide toehold region at each end of the bridge sequence the proximity probes react with the toehold regions; simultaneous toehold mediated displacement occurs between the proximity probes and the bridge sequence in the presence of a target protein thereby releasing the reporter sequence; the reporter sequence activates a target recycling amplification process (TRAP) through strand displacement reactions thereby tethering an AuNP probe; and the imaging platform is configured to visualize and quantify the tethered AuNP probes using photonic resonator adsorption microscopy (PRAM).

In one aspect, the disclosure provides a system wherein the sample contains the target protein.

In one aspect, the disclosure provides a system wherein the sample does not contain the target protein.

In one aspect the disclosure provides a system, wherein the toehold region is comprised of at least six exposed nucleotides.

In one aspect the disclosure provides a system, wherein the proximity probes are created through antibody oligonucleotide conjugation to form DNA reactive affinity targets.

In one aspect the disclosure provides a system, wherein the DNA reactive affinity targets can react with the toehold regions.

In one aspect the disclosure provides a system, wherein the bridge-reporter duplex is stable in the presence of one or both proximity probes and the reporter sequence is stable.

In one aspect the disclosure provides a system wherein the presence of a target protein causes simultaneous toehold-mediated displacement reactions between the proximity probe and the bridge sequence thereby releasing the reporter sequence.

In one aspect the disclosure provides a system, wherein the released reporter sequence is a single stranded DNA (ssDNA).

In one aspect the disclosure provides a system, wherein the released reporter sequence activates the TRAP.

In one aspect the disclosure provides a system, wherein TRAP allows for a single reporter to bind multiple AuNP to the photonic crystal for quantification.

In one aspect the disclosure provides a system, wherein the system can be used to identify post-translational modifications or protein-protein interactions.

In one aspect the disclosure provides a biological assay, comprising a bridge sequence, a reporter sequence, two proximity probes, a solution; and one or more adapters each tethered to an antibody, and a sample; wherein the bridge sequence and reporter sequence are annealed to form a stable bridge-reporter duplex with an exposed toehold region at each end of the bridge sequence and the proximity probes bound to an antibody-linked adapter can bind a target antigen.

In one aspect the disclosure provides a biological assay wherein the sample contains the target protein.

In one aspect the disclosure provides a biological assay, wherein the sample does not contain the target protein.

In one aspect the disclosure provides a biological assay, wherein the stable duplex has an exposed toehold region consisting of 2-15 nucleotides on each end.

In one aspect the disclosure provides a biological assay, wherein the toehold region is comprised of at least six exposed nucleotides.

In one aspect the disclosure provides a biological assay, wherein the proximity probes are created through antibody oligonucleotide conjugation to form DNA reactive affinity targets.

In one aspect the disclosure provides a biological assay, wherein DNA reactive affinity targets can react with the toehold regions.

In one aspect the disclosure provides a biological assay, wherein the bridge-reporter duplex is stable in the presence of one or both proximity probes and the reporter sequence is stable.

In one aspect the disclosure provides a biological assay, wherein the presence of a target protein causes simultaneous toehold-mediated displacement reactions between the proximity probe and the bridge sequence thereby releasing the reporter sequence.

In one aspect the disclosure provides a biological assay, wherein the released reporter sequence is a single stranded DNA (ssDNA).

In one aspect the disclosure provides a biological assay, wherein target recycling amplification process (TRAP) and photonic resonator adsorption microscopy (PRAM) are used to quantify protein expression via quantification of gold nanoparticles (AuNP).

In one aspect the disclosure provides a method for detecting and/or quantifying proteins in a sample, comprising the steps of annealing a bridge sequence and a reporter sequence to form a stable bridge-reporter duplex with a six-nucleotide exposed toehold region at each end of the bridge sequence; simultaneous toehold displacement of one or more proximity probes and the bridge sequence in the presence of a sample containing a target protein thereby releasing a reporter sequence; activation of a target recycling amplification process (TRAP) through strand displacement reactions thereby tethering an AuNP probe; and visualizing and quantifying tethered AuNP using photonic resonator adsorption microscopy (PRAM).

In one aspect the disclosure provides a method, wherein the sample contains the target protein.

In one aspect the disclosure provides a method, wherein the sample does not contain the target protein.

In one aspect the disclosure provides a method, wherein the proximity probes are created through antibody oligonucleotide conjugation to form DNA reactive affinity targets.

In one aspect the disclosure provides a method, wherein DNA reactive affinity targets can react with the toehold regions.

In one aspect the disclosure provides a method, wherein the bridge-reporter duplex is stable in the presence of one or both proximity probes and the reporter sequence is stable.

In one aspect the disclosure provides a method, wherein the presence of a target protein causes simultaneous toehold-mediated displacement reactions between the proximity probe and the bridge sequence thereby releasing the reporter sequence.

In one aspect the disclosure provides a method, wherein the released reporter sequence is a single stranded DNA (ssDNA).

In view of the present disclosure, the methods and compositions described herein can be configured by the person of ordinary skill in the art to meet the desired need.

### 1. Overview

The current disclosure provides an improved sensitivity assay, for detecting and quantifying proteins in a sample. The disclosure also provides a system and method for use of the assay. The assay utilizes target recycling amplification process (TRAP) for the digital detection of proteins in tandem with photonic resonator absorption microscopy (PRAM) and utilizes toehold-mediated DNA strand displacement reactions. The disclosed assay, system, and methods provide enhanced sensitivity to detect and quantify nucleic acids in a biological sample and can be used in numerous settings including health status, early disease and medical condition diagnosis, identification of biomarkers, evaluation of therapeutic efficiency, and longitudinal monitoring of disease progression. The PRAM instrument is described in U.S. patent application Ser. No. 16/170,111 while various aspects of photonic crystal (PC) biosensors are described in U.S. Pat. Nos. 7,479,404, 7,521,769, 7,531,786, 7,737,392, 7,742,662, and 7,968,836, all of which are incorporated herein by reference.

### 2. Photonic Resonator Absorption Microscopy (PRAM)

Photonic resonator absorption microscopy (PRAM) utilizes gold nanoparticles (AuNPs) bound to a photonic crystal surface that can be visualized and counted. PRAM utilizes digital detection with a spectrometer-based PRAM, and to achieve rapid detection (1 hour) of HIV p24 antigen (LOD of 1 pg/mL) and COVID-19 IgG (LOD of 27 pg/mL) using a sandwich assay format. PRAM permits a single antigen to bind a single nanoparticle (one-to-one), thereby consuming the protein target. The binding is recognized via digital detection allowing for increased sensitivity.

PRAM can visualize individual gold nanoparticle (AuNPs) tags on a photonic crystal (PC) surface through resonance coupling, in addition to the alternative tags disclosed herein. The detection principle of PRAM utilizes the resonant PC reflection at a wavelength of λ=625 nm to provide a high reflected intensity from collimated low intensity LED illumination of the same wavelength into a webcam-variety image sensor. More specifically, Port 1 is coupled to a fiber-coupled 617 nm LED light source (M617F2, Thorlabs), and a lens group (F810SMA-635, Thorlabs) is first utilized to collimate the output beam. A zero-order half-wave plate (WPH10M-633, Thorlabs) rotates the polarization of the collimated beam in order to excite the TM resonance mode of the PC cavity. A plano-convex lens (LA1509-A-ML, Thorlabs) then focuses the beam onto the back focal plane of an Olympus plan-fluorite objective 20×/0.5 numerical aperture (NA) objective, from which a collimated beam impinges onto the PC surface at normal incidence. A manual three-axis stage (PT3, Thorlabs) is used to secure the PC sample at the focal plane of the objective. The reflected light from the PC resonator is the collected by the same objective and redirected by a 50/50 non-polarizing beam-splitter (CCM1-BS013, Thorlabs). A doublet (AC254-200-A-ML, Thorlabs) projects the image plane onto a charge coupled device (CCD) camera (GS3-U3-51S5M-C, Point Grey), with a resolution of 177 nm/pixel. At a particular resonant wavelength and incident angle, complete interference occurs, and no light is transmitted, resulting in nearly 100% reflection efficiency. The resonant reflectance magnitude is dramatically reduced by the addition of absorbing AuNPs upon the PC surface, resulting in the ability to observe each AuNP by illuminating with light from an LED and making images of the reflected intensity.

By measuring the resonant peak intensity value (PIV) on a pixel-by-pixel basis across the PC using a microscopy, the output of PRAM is PIV images of attached AuNPs. The images may be gathered by illuminating the structure with collimated broadband light through the transparent substrate, while the front surface of the PC is immersed in aqueous media. The AuNPs are strategically selected to provide strong absorption by localized surface plasmon resonance at the same wavelength. Thus, each surface-bound AuNP registers in the PC reflected image as a location with reduced intensity, compared to the surrounding regions without AuNPs. By immobilizing target-activated AuNP probes on a PC surface, PRAM has been used to quantify nucleic acids and proteins with single-particle resolution. Previous work focused on detecting chemically synthetic miRNAs with similar detection limits to qtr.-PCR methods, in which each detected miRNA molecule was associated with one AuNP tag. The current disclosure adds the technical effect of providing an amplification mechanism that further reduces detection limits, as the target nucleic acid molecule is consumed by the detection process. This is achieved through use of DNA-fueled molecular machines, which are cascade DNA circuits that are driven by fuel strands (in TRAP, for example, the miRNA target and the probe sequence act as a fuel) where the whole process cycles due to entropy. The DNA-fueled molecular machines include a series of toehold-mediated DNA strand displacement reactions involving target recycling, as tools for building switchable nanodevices, controlled nanoparticle assembly, mediated gene expression, and programmed DNA computation. This results in the technical effects of a >400-fold reduction in miRNA detection limits, into sub-attomolar concentrations and decreasing the assay time to 20 minutes using PRAM detection in conjunction with target recycling by a DNA-fueled molecular machine. Furthermore, PRAM couple with TRAP offers the additional technical effects of a single step, room temperature, single vessel reaction requiring only synthetic nucleic acids with a low total cost per sample tested.

### 3. Target Recycling Amplification Process (TRAP)

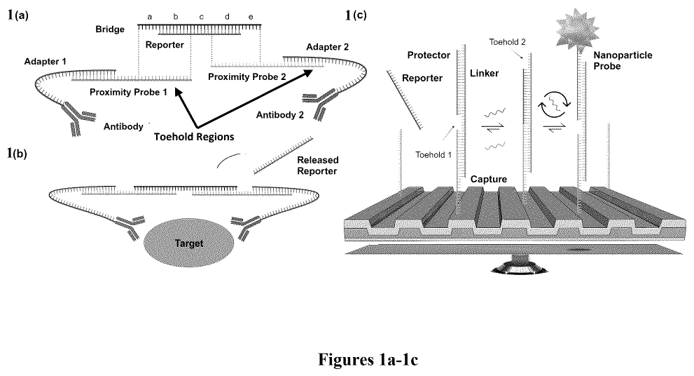

As used herein “Target Recycling Amplification process (TRAP)” refers to a method that allows for amplification of a nucleic acid signal to increase the sensitivity of biosensors, assays for biomarker detection, and other types of nucleic acid detection. In an initial step of the current disclosure, miRNA is extracted from exosomes isolated from cell culture media (FIG. 1a) using a variety of commonly known protocols in the art. A photonic crystal (PC) surface is then prepared with an immobilized capture DNA or RNA through any DNA or RNA mobilization process. Examples of RNA and DNA mobilization processes include, but are not limited to covalent bonding methods, streptavidin-biotin methods, PEG linkers, and self-assembled monolayer linkers. The photonic crystal has previously been described in U.S. Pat. Nos. 7,479,404, 7,521,769, 7,531,786, 7,737,392, 7,742,662, and 7,968,836, all of which are incorporated herein by reference.

Following immobilization of the capture DNA the PC is pretreated with a linker-protector complex consisting of a protector DNA and a linker DNA. The linker-protector complex is designed with free unhybridized regions at both 5′ and 3′ ends of the linker strand, resulting in formation of toehold-1 and toehold-2 for miRNA or other nucleic acid binding and probe DNA binding, respectively.

TRAP can be used for microRNA detection and is advantageous as it reduces assay times from 2 hours to 10 minutes, when compared to a previous one-to-one AuNP binding assay for microRNA. TRAP also demonstrates improved limits of detection from 100 aM to 0.5 aM versus previous one-to-one AuNP binding assay for microRNA. TRAP is designed so that a single nucleic acid target sequence is recycled through multiple toehold-mediated strand displacement reactions to bind a nanoparticle onto the PC for counting. Thus, the target is never consumed, linearly amplifies the signal. TRAP has a 60-fold lower limit of detection for microRNA when compared to PCR. TRAP does not require exponential amplification, instead the “amplify-then-digitalize” approach allows rapid, ultrasensitive, room temperature, enzyme-free detection that rivals traditional amplification techniques. Improvements in the photonic resonator absorption microscope, allows for generation of a portable benchtop version costing less than $10,000 while still allowing for accurate digital counting of nanoparticles.

As used herein “protector DNA or protector” refers to a nucleic acid or nucleic acid sequence that is not functional in the system but keeps unintended DNA from reacting. One of skill in the art will readily understand the role of “protector DNA”.

As used herein “linker DNA” or “substrate” or “bridge” refer to a DNA sequence used to link the captured label to the detection substrate. In some embodiments, the linker works via hybridization of both the capture and protector DNAs.

As used herein “Toehold Mediated Strand Displacement (SDR) or toehold-mediated branch migration reaction or entropy-driven toehold SDR” refers to an enzyme-free molecular method by which one strand of a nucleic acid (output) is exchanged with another nucleic acid strand (input). More particularly, the terms refer to any system where a duplex nucleic acid initially has a short toehold sequence of 4-8 nucleotides. In one embodiment the sequence is single stranded. An invading strand binds to the toehold, and a branch migration reaction occurs between the three strands allowing the invading strand to displace the protector strand. Systems wherein toehold is hidden until exposed by another reaction, such as in TRAP, are called “toehold sequestering” and can also be part of the disclosure herein. In an exemplary embodiment the toehold reactions occur via addition of reaction solutions, commonly known in the art, to a Polydimethylsiloxane (PDMS) reservoir attached to the PC surface. The reservoirs can also be comprised of a wide range of materials including, but not limited to, plastic (such as acrylic, polycarbonate, polyester), glass.

### 4. Proximity Immunoassay with Nucleic Acid Target Amplification (PINATA)

As used herein “Proximity Immunoassay with Nucleic Acid Target Amplification (PINATA)”, refers to a method that combines non-enzymatic amplification with digital detection for a room temperature, no-wash, single-step assay characterized by rapid and ultrasensitive protein quantification. The PINATA assay is designed to transduce a target protein into a short single-stranded DNA reporter sequence using two antibody-oligonucleotide conjugates (AbOs) and dual toehold-mediated strand

PINATA utilizes the principle of DNA molecular circuits, as used in DNA computing applications, such as the creation of simple DNA logic gates. PINATA operates on the logic of using a AND/OR Boolean signal response depending on whether one or two specific DNA sequences are present. PINATA exploits the creation of the AND gate, where an action only occurs driven by the proximity of the two probes. Employing two simultaneous toehold-mediated strand displacement reactions allows the conversion of a protein into a ssDNA reporter sequence by using proximity probes. The reporter is then linearly amplified and detected using the TRAP approach with photonic resonator absorption microscopy. In this manner, PINATA is a novel and inventive non-enzymatic amplification method with digital detection for a room temperature, no-wash, single-step assay. PINATA allows for rapid and ultrasensitive protein quantification.

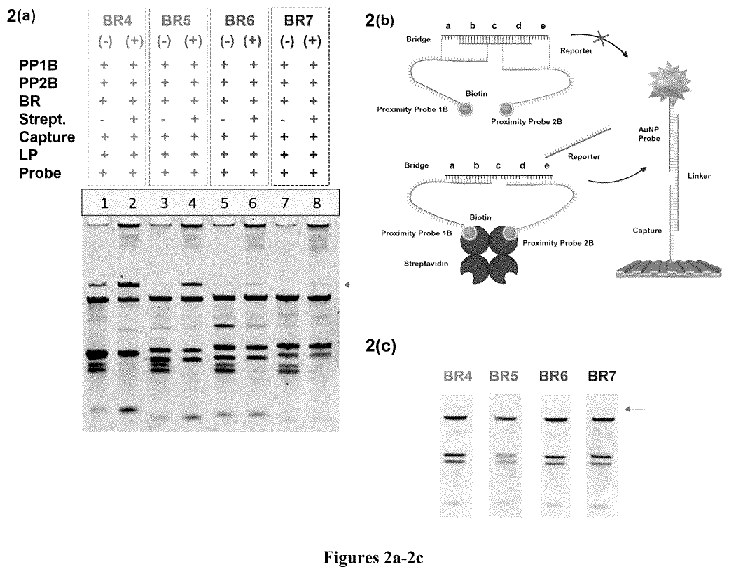

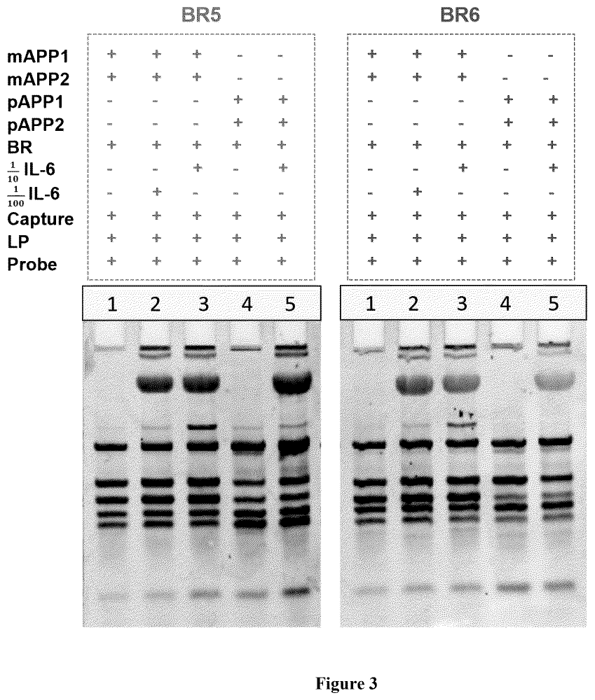

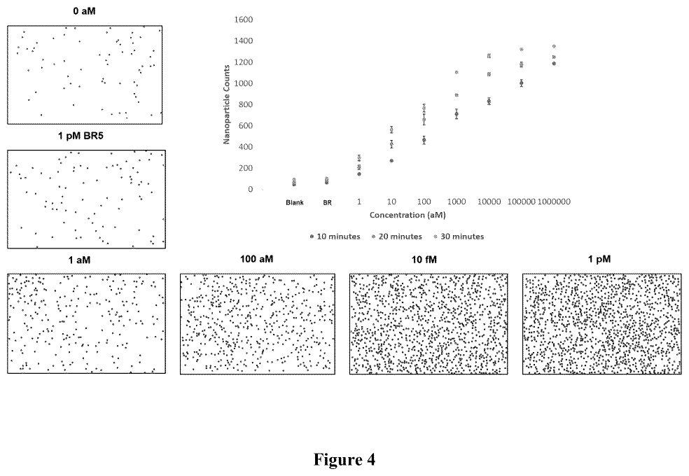

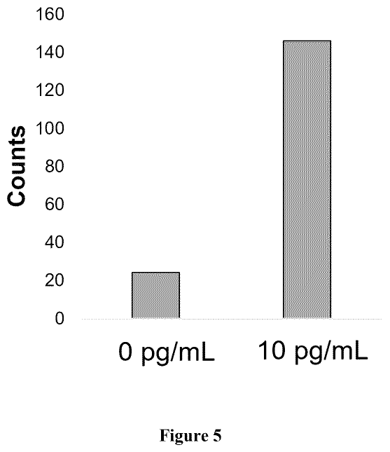

The PINATA assay transduces a target protein into a short single-stranded DNA reporter sequence using two antibody-oligonucleotide conjugates (AbOs) and dual toehold-mediated strand displacement reactions. In a preferred embodiment, the bridge and reporter sequences (FIG. 1a) are initially annealed to form a stable duplex, with six-nucleotide exposed toehold regions on either end (FIG. 1a; regions a and e). Proximity probes are created through antibody-oligonucleotide conjugation to create DNA-reactive affinity targets that can react with the toehold regions (FIG. 1a). Alone, or in the presence of one or both proximity probes in solution, the bridge-reporter duplex is stable, and no reporter will be displaced, and further amplification is not triggered (FIG. 2b). When both proximity probes are brought into close vicinity by a target protein, the simultaneous toehold-mediated displacement reactions between the proximity probes and the bridge sequence cause the reporter to be released (FIG. 1b). The ssDNA reporter sequence then activates target recycling through additional strand displacement reactions, allowing a single reporter to bind multiple nanoparticles (AuNP) to a photonic crystal for digital counting (FIG. 1c). The reporter initially interacts with an exposed toehold region on the linker strand, displacing the protector strand. This exposes a second toehold region on the linker that was initially sequestered, allowing the NP probe sequence conjugated to an AuNP to begin a second toehold-mediated strand displacement reaction. This displaces the reporter, allowing a single reporter to enact target recycling to bind many nanoparticles. If no reporter is released into the system, no nanoparticles can bind, indicating there is no target protein.

In one aspect of this disclosure PINATA is used with additional standard enzymatic and non-enzymatic amplification techniques. The approach can be applied to various other reporting systems and labels. In one aspect, the digital and non-digital detected labels include quantum dots, fluorophores, plasmonic fluors, magnetic nanoparticles or beads, electrochemical sensors, barcoding, or various other optical sensing methods. In one aspect, digital detection methods achieve the highest sensitivity, but speed or ease is improved by using bulk signal detection.

In one aspect of this disclosure, PINATA is adapted to work with affinity groups for the detection of different targets including, but not limited to, antibodies, aptamers, nanobodies, and other affinity groups. Such affinity groups could be applied for use in protein detection and quantification.

In one aspect of this disclosure, assays are used to study protein-protein interactions and kinetics, or various posttranslational modifications. Using a single DNA sequence-selective proximity probe paired with an affinity probe, the assay is used to examine protein-RNA or protein-DNA interactions.

In one aspect of this disclosure, the assays are used in methods that require ultra-sensitivity like the detection of circulating tumor cells or tissue specific exosomes.

In one aspect of this disclosure, when using two DNA sequence-specific proximity probes, the same assay is applied to detect nucleic acid targets that could be particularly aided by the requirement of proximity annealing, such as messenger RNA splice variants, circular RNAs, or modified RNA sequences. Overall, the sensitivity limits and rapid assay times are novel and inventive aspects of PINATA as a powerful tool for studying rapid and ultrasensitive protein quantification. PINATA further demonstrates the unexpected advantages of application of the method at room temperature assay conditions, linearly amplified signal, and the sensitivity of digital detection for studying various affinity targets.

As used herein “ultrasensitive” refers to the detection limits of the PINATA methods. Ultrasensitive protein detection limits are in the attomloar to femtomolar range or below.

The system, assay, and method of the current disclosure have the technical effect of being stable at room temperature. This overcomes a significant limitation of current assays and systems, which require cold storage. Further, the system, assay, and method provided herein have the additional surprising technical effect of providing a result in less than twenty minutes allowing for rapid assessment of health status, early disease and medical condition diagnosis, identification of biomarkers, evaluation of therapeutic efficiency, and longitudinal monitoring of disease progression. As such the system, assay, and method described herein can be used at the point of care.

### Examples

The Examples that follow are illustrative of specific embodiments of the invention, and various uses thereof. They are set forth for explanatory purposes only and should not be construed as limiting the scope of the disclosure in any way.

### Materials and Methods

### Materials

### Proteins and Antibodies

Pierce Streptavidin (PI21122) was ordered from Thermo Scientific (Waltham, MA). Polyclonal antibodies, monoclonal antibodies, and ELISA (enzyme linked immunosorbent assay) kit were purchased from R&D Systems (Minneapolis, MN). The polyclonal antibody tested is a goat IgG Human IL-6 Antibody (AF-206-NA). The monoclonal pairs tested were mouse-derived Human IL-6 MAb Clone 1936 (MAB2061) and recombinant rabbit-derived Human IL-6 MAb Clone 2828D (MAB95402). The R&D DuoSet Human IL-6 ELISA kit (DY206-05) was used for comparison, which uses the same monoclonal antibodies. The human IL-6 standard used for testing was also from the DuoSet ELISA kit.

### Oligonucleotide-Antibody Conjugation Kits

Oligonucleotide Conjugation Kits (ab218260) purchased from Abcam were used to attach antibodies to oligonucleotide adapters. Adapter oligonucleotide sequences were purchased from IDT as either 5′ or 3′ amine-terminated and purified through HPLC.

### Oligonucleotide-Antibody Conjugation for Human IL-6 Detection

Oligonucleotide conjugation kits were used to attach 5′ and 3′ amine-terminated HPLC purified adapter oligonucleotides to monoclonal and polyclonal antibodies. Antibodies were bound to at least one DNA molecule, and to avoid multiple sequences attaching to multiple locations on the antibodies, the DNA/antibody ratio used was 1.5 with a 40 pM concentration of DNA. The adapter oligonucleotides were hybridized to the chosen proximity probe in saturation, and excess proximity probe sequences were removed using magnetic Dynabeads.

### Photonic Crystals:

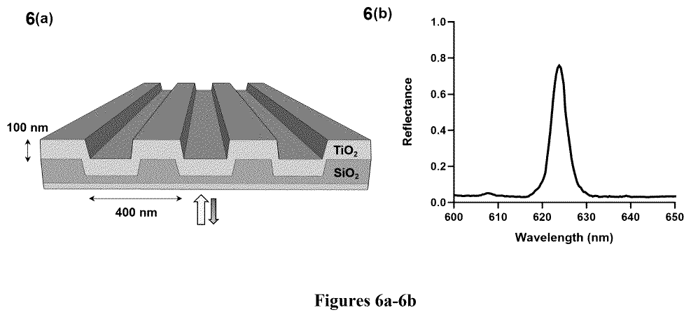

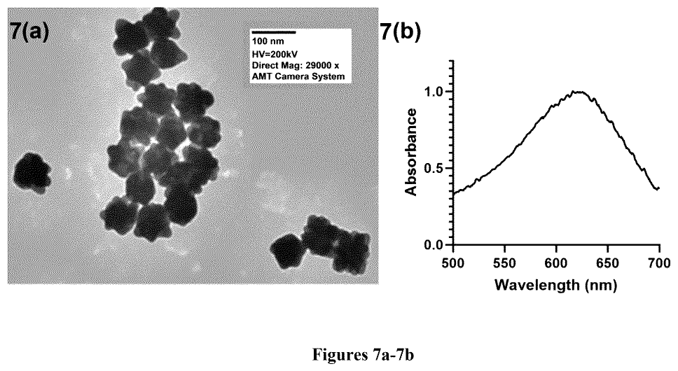

All photonic crystals (PCs) used were purchased from Moxtek (Orem, UT). The PC structure is composed of a low refractive glass grating structure with a period of 280 nm coated with higher refractive titanium dioxide, which are fabricated on 8-inch glass wafers with a 10 nm etch stop layer of Al2O3, followed by depositing a layer of SiO2 then using large area ultraviolet interference lithography to form the one-dimensional grating structures. A thin layer of TiO2 with a thickness of 100 nm is then deposited on the etched 8-inch wafers, which are then cut to smaller sizes of 1×1.2 cm. PCs are glued to cover slips with optically clear UV-curing adhesive (Norland Optical Adhesive 63) for easy use with microscope. To form wells, polydimethylsiloxane (PDMS) was applied to the top of the PCs using light pressure, with 2.5 mm diameter wells punched through to allow solutions in the wells to contact the PC surface. PC spectra were tested for all PCs used in experiments and showed a photonic-coupled grating resonance (PCGR) for high reflectivity between 620-625 nm wavelengths of light when wells were filled with 1×TE buffer solution (FIG. 6).

### Nanoparticles:

Commercial spiky 80 nm gold NanoUrchins (NPs) were purchased from Cytodiagnostics for their high absorption of red light from 610-630 nm wavelengths.

Nucleic acids: All DNA sequences were purchased from Integrated DNA Technologies (Coralview, IA), with standard desalting purification. Amine-modified and thiol-modified sequences for conjugation onto photonic crystals and gold nanoparticles, respectively, included a 15-nucleotide poly-T spacer, chosen to reduce reactivity with the gold nanoparticle surface to prevent absorption.1 All sequences used are listed in Table 1. Concentrations of oligonucleotides were established using a Nanodrop One C UV/Vis spectrometer to calculate molar extinction coefficient of single-stranded DNA.

TABLE 1
Oligonucleotide sequences names, abbreviations,
compositions, and modifiers for conjugation to
antibodies, nanoparticles, and photonic crystals.

| SEQ | | | |
|---|---|---|---|
| ID | Oligo | | Sequence (5′->3′) and |
| NO. | Name | Abbr | Modifications |
| 1 | Proximity | PP1B | CTAAGTATCATATAGTTTTTTTTTTTTTTT |
| | probe 1 - | | TTTTTTTTTT/3Bio/ |
| | biotin | | |
| 2 | Proximity | PP2B | /5Biosg/TTTTTTTTTTTTTTTTTTTTTT |
| | probe 2 - | | TTTCGTGATGTAATCTGC |
| | biotin | | |
| 3 | Adapter 1 | A1 | (HPLC) |
| | | | /5AmMC6/TTTTTTTTTTTTTTTCAGTATG |
| | | | AAGTGAACTAGTC |
| 4 | Adapter 2 | A2 | (HPLC) |
| | | | CATTCTAGAGATTGTGACTATTTTTTTTTT |
| | | | TTTTT/3AmMO/ |
| 5 | Proximity | PP1 | CTAAGTATCATATAGTTTTTTTTTTTTTTT |
| | probe 1 | | TTTTTGACTAGTTCACTTCATACTG |
| 6 | Proximity | PP2 | TAGTCACAATCTCTAGAATGTTTTTTTTTT |
| | probe 2 | | TTTTTTTTTTCGTGATGTAATCTGC |
| 7 | Bridge 4 | B4 | CTATATGATACTTAGTGTAGCAGATTACAT |
| | | | CACG |
| 8 | Bridge 5 | B5 | CTATATGATACTTAGTTGTAGCAGATTACA |
| | | | TCACG |
| 9 | Bridge 6 | B6 | CTATATGATACTTAGTTGTATGCAGATTAC |
| | | | ATCACG |
| 10 | Bridge 7 | B7 | CTATATGATACTTAGTTGTATGGCAGATTA |
| | | | CATCACG |
| 11 | Reporter 4 | R4 | GTAATCTGCTACACTAAGTAT |
| 12 | Reporter 5 | R5 | GTAATCTGCTACAACTAAGTATC |
| 13 | Reporter 6 | R6 | GTAATCTGCATACAACTAAGTATC |
| 14 | Reporter 7 | R7 | GTAATCTGCCATACAACTAAGTATC |
| 15 | Linker 4 | L4 | CAATAACTACTCAATCTCGATACTTAGTGT |
| | | | AGCAGATTACCAACTC |
| 16 | Linker 5 | L5 | CAATAACTACTCAATCTCGATACTTAGTTG |
| | | | TAGCAGATTACCAACTC |
| 17 | Linker 6 | L6 | CAATAACTACTCAATCTCGATACTTAGTTG |
| | | | TATGCAGATTACCAACTC |
| 18 | Linker 7 | L7 | CAATAACTACTCAATCTCGATACTTAGTTG |
| | | | TATGGCAGATTACCAACTC |
| 19 | Protector 4 | P4 | GTTGGTAATCTGCTACACTAA |
| 20 | Protector 5 | P5 | GTTGGTAATCTGCTACAACTAA |
| 21 | Protector 6 | P6 | GTTGGTAATCTGCATACAACTAA |
| 22 | Protector 7 | P7 | GTTGGTAATCTGCCATACAACTAA |
| 23 | NP probe 4 | Pr4 | /5ThioMC6-D/ |
| | | | TTTTTTTTTTTTTTTGAGTTGGTAATCTGC |
| | | | TACACTAA |
| 24 | NP probe 5 | Pr5 | /5ThioMC6-D/ |
| | | | TTTTTTTTTTTTTTTGAGTTGGTAATCTGC |
| | | | TACAACTAA |
| 25 | NP probe 6 | Pr6 | /5ThioMC6-D/ |
| | | | TTTTTTTTTTTTTTTGAGTTGGTAATCTGC |
| | | | ATACAACTAA |
| 26 | NP probe 7 | Pr7 | /5ThioMC6-D/ |
| | | | TTTTTTTTTTTTTTTGAGTTGGTAATCTGC |
| | | | CATACAACTAA |
| 27 | PC capture | C | GAGATTGAGTAGTTATTGTTTTTTTTTTTT |
| | | | TTT /3AmMO/ |

All particle counting images were taken with the portable Photonic Resonator Absorption Microscope (PRAM), with an automated motion capture stage, camera, 50× objective, and 633 nm LED that couples with the photonic crystal guided resonance mode. Dynamic light scattering (DLS) and zeta potential data were collected from the Malvern Zetasizer. Fluorescence data was collected from QuantStudio.

### Methods

### Oligonucleotide Sequence Design:

Oligonucleotides were designed using the online NUPACK software analysis tool to minimize secondary structures and cross-reactivity. The bridge-reporter sequences were chosen by initial testing of different toehold and tuning lengths of nucleotides that are not displaced by proximity probes to minimize the background signal when no target was present and to maximize the amount of reporter displaced when the proximity probes are brought into close contact by a target protein. Each proximity probe was designed to be tested with a variety of different bridge-reporter lengths.

### Magnetic Bead Duplex Purification of Bridge-Reporter Duplex and Proximity Probes:

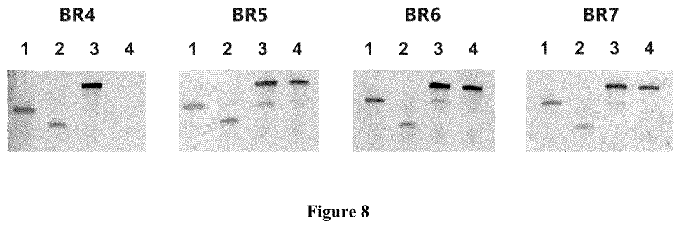

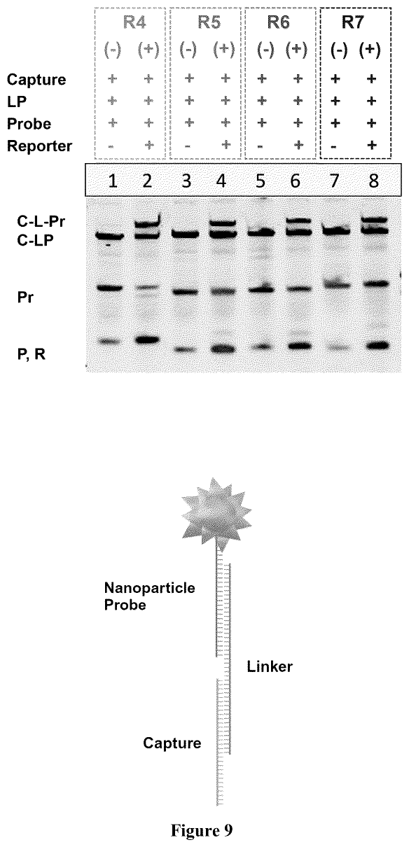

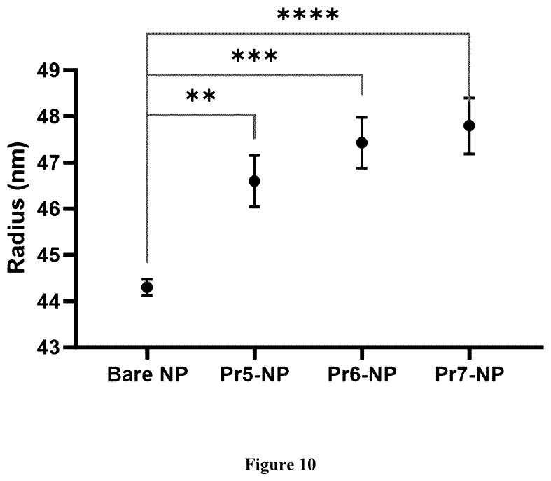

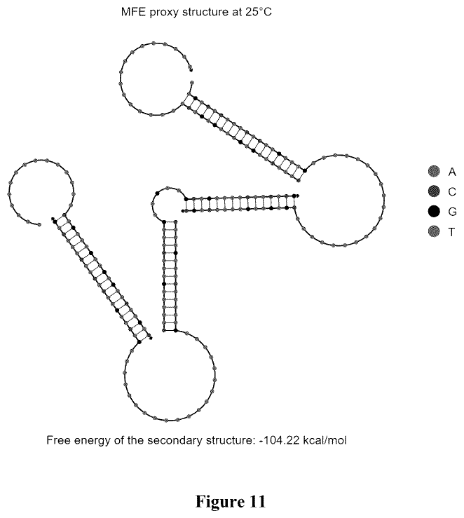

The bridge-reporter duplexes and the antibody-adapter-proximity probe complex were purified using streptavidin coated magnetic Dynabeads. The bridge-reporter duplexes were initially annealed at a 1:1.2 ratio with bridge in excess. The excess bridge sequences were then purified using Streptavidin MyOne C Dynabeads conjugated to the biotinylated BR purifier for that pair (sequences shown in Table 1). Each bridge sequence hybridizes to its corresponding reporter sequence, i.e., B4 and R4 would be annealed to create the BR4 duplex. To anneal, oligonucleotides were diluted to 5 μM reporter and 6 μM bridge in a pH 7.4 1×TE, 12.5 mM MgCl2 buffer. This resulted in a reaction with excess bridge without displacing the reporter on properly annealed bridge-reporter duplexes, as neither toehold region is activated, The biotinylated DNA purifier sequence was saturated onto the beads according to the Dynabeads standard procedure using a biotin-streptavidin reaction and washed four times using a magnetic stand to remove excess unbound purifiers. Beads (100 μL) were reacted with BR duplex (300 μL) for 1 hour at room temperature. Bridge sequences not annealed to a reporter were removed using a magnet. The process was verified by native PAGE. Assay probe purification was repeated three times. Excess proximity probes from the antibody-oligonucleotides conjugates that did not bind to the adapter sequence were purified using same method, and proximity probe purifier sequences shown in Table 1. The process was verified by native PAGE results (FIG. 8).

### Linker-Protector Annealing:

Linker-protector sequences are annealed together with a stoichiometric ratio of 1:1.5 with the protector sequence in excess, at a 5 μM linker concentration in a 1×TE, 12.5 mM MgCl2 buffer, by heating sequences to 90° C. for 5 minutes, then slowly allowing to cool to room temperature. LP duplexes were stored at 4° C. for short term use or aliquoted and frozen at −20° C.

### Native PAGE:

All native PAGE procedures with DNA were loaded with non-SDS 6× loading dye (New England Biolabs), run on the Biorad Mini-PROTEAN Tetra system in a 1×TBE running buffer, stained with Gel Red (Gold Bio), and imaged using the Gel Doc XR imaging system.

### Nucleic Acid Target Recycling Amplification Process Feasibility in Native PAGE:

To verify the target recycling amplification process of the reporter sequence, each reporter sequence was tested with its respective probe, linker, and protector sequences. The same capture sequence was used for all four reporters. The capture, linker-protector, and probe sequences, with reporter added for the positive control, were initially reacted at a 100 nM concentration for 2 hours at room temperature in 0.5×PBS, 0.5×TE, 5 mM MgCl2 buffer, then run on a 15% native PAGE. The final capture-linker-probe triplex, which represents an AuNP binding to the PC, appears in only the positive lanes when the reporter was added, indicating the success of toehold-mediated strand displacement amplification. The annealed and purified bridge-reporter sequence was also tested to see if it causes amplification.

Biotin-Streptavidin Testing with Native Polyacrylamide Gel Electrophoresis (PAGE):

DNA sequences were tested using proximity probes terminated with 5′ or 3′ biotin modifications to detect streptavidin (Table 1). This mimic system of protein-antibody binding allowed more cost-effective initial testing of many potential sequences, without requiring oligonucleotide-antibody conjugation kits. To test each bridge-reporter duplex, the pre-hybridized purified bridge-reporter duplex, and stoichiometric equivalents of PP1B and PP2B were mixed in a 0.5×PBS, 0.5×TE, 5 mM MgCl2 buffer. For a positive control, one equivalent of streptavidin was added as well. These sequences reacted for 30 minutes at room temperature to ensure biotin-streptavidin binding, then the capture, linker-protector and probe sequences for each bridge-reporter length were added. All sequences were then incubated for an additional 1 hour at room temperature, then samples were run in a 12% native PAGE.

### Gold Nanoparticle-Oligonucleotide Conjugation (AuNP-Pr):

Gold nanoUrchins (80 nM) were initially washed by centrifugation for 30 minutes at 600 rcf, and the supernatant removed, and the process repeated three times. AuNPs were concentrated to 1 OD, or ˜7.8 billion NPs/m, and stored in a 0.01×PBS buffer until conjugation. Thiol-modified DNA were reduced using 100 equivalents of TCEP for two hours at room temperature, and excess TCEP and trityl-SH products were removed using a 0.5 mL 3K Amicon centrifuge filter via centrifugation at 14.1 rcf for 15 minutes and repeated three times. The reduced DNA-SH probe sequences were then added to the AuNPs at a final concentration of 100 nM. After 15 minutes, 1k mPEG-SH (Creative PegWorks) at 40 μg/mL concentration was added as well for surface blocking and incubated for two days at room temperature to self-assemble on the gold nanoparticles. Unreacted probe DNA and PEG were then removed through centrifugation washing three times at 600 rcf for 30 minutes. Conjugated AuNP-Pr was resuspended at a final OD of 2 in 1×TE, 0.025% tween20, and stored at 4° C. for up to four weeks or until use. Properly conjugated AuNPs showed a 1-2 nm increase in radius demonstrated a change in the absorbance spectra.

### Photonic Crystal DNA Capture Surface Functionalization:

Photonic crystals (PC) were prepared for silanization by sonicating for two minutes in acetone, isopropanol, and MilliQ® water. The PCs were dried under nitrogen gas and then heated to 120° C. to completely dry. The PCs were oxygen plasma-treated to create reactive hydroxylated surface using PicoDiener plasma machine at 0.9 mbar and 100% power. PCs were then immediately added to a 2% (3-Aminopropyl) triethoxysilane (APTES, 99% purity) solution in 95% ethanol and 5% water (% v/v) for 1 hour at room temperature, removed and washed by sonicating with acetone, ethanol, and then MilliQ® water for one minute each. Silanized PCs were dried gently under nitrogen gas, and cured for an hour at 80° C. and used immediately for capture attachment or stored in a desiccator under nitrogen gas. PDMS well gaskets were attached through light pressure to the PC surface. Disuccinimidyl carbonate (DSC) was used as a bifunctional linker between APTES and amine-terminated capture DNA and was added to the PC in the wells at 10 mM in 10% DMSO for 20 minutes at room temperature. The wells were washed with 1% DMSO, 0.1% DMSO, and water before capture DNA was added at 50 μM for three hours at room temperature.

Nucleic Acid Target Amplification Testing with PRAM:

Capture-functionalized photonic crystals were washed five times with a 1×TE, 0.025% tween20 washing buffer after capture incubation. PCs were blocked using Superblock for 30 minutes at room temperature and washed twice. Annealed LP duplexes (final concentration of 20 μM), nanoparticle AuNP-Pr (final OD=0.5), and magnesium chloride (final concentration 5 mM), along with the tested reporter concentrations (0 aM, 0.1 aM, 1 aM, 10 aM, 100 aM, 1 fM, 10 fM, 100 fM, and 1 pM) were added to the 20 μL-containing well on the PC surface. PRAM images were captured at 10, 20 or 30 minutes.

### PRAM Image Processing and Nanoparticle Counting:

The PRAM nanoparticle images were filtered, analyzed, and counted using MATLAB. A Tophat filter and a Fourier DC component removal algorithm were applied to eliminate uneven background, speckles, and fringes induced by multi-surface reflection or dust diffraction. The noise of the image was suppressed through a simple Wiener filter. An extremum searching algorithm using maximally stable extremal regions (MSER) was applied to recognize AuNPs. AuNP features including the threshold of gray scale, size, and circularity, Euler number, and equivalent diameters were considered as criteria to seek the extreme regions generated from AuNPs. To clearly visualize the particle counting process, the detected AuNPs were marked with identical circles on a blank background at their corresponding centroid location.

### Results

### Design and Optimization of the PINATA Assay:

Streptavidin and biotinylated DNA sequences were used as a protein-antibody mimic to assess a set of four different sequence options, (e.g. 4, 5, 6, and 7), wherein the number refers to the number of nucleotides on the bridge-reporter sequence in middle tuning region “c” (FIG. 2b) that are not displaced by the proximity probes. “BR4” refers to a bridge-reporter duplex with a 4-base region that is not displaced, and “BR7” refers to a bridge-reporter sequence with a 7-base region. When no streptavidin is present, the reporter does not release from the bridge and cannot amplify. When streptavidin brings the biotinylated proximity probes together, the reporter sequence is displaced, and then amplified for AuNP capture and counting. To increase the rate of reporter release, the tuning region of the bridge-reporter duplex was shortened, which makes it more thermodynamically favorable for reporter release to occur. To decrease the rate of release, the tuning region was lengthened, creating more paired nucleotides that are not displaced by either proximity probe, strengthening the stability of the bridge-reporter duplex. There was obvious amplification when no streptavidin was present for BR4 set (FIG. 2a, Lane 1), indicating that the proximity probes were undergoing dual strand displacement reactions in solution. There was also an extremely low positive signal response to streptavidin for BR7 (FIG. 2a, Lane 8), so BR4 and BR7 were eliminated from further testing. BR5 and BR6 both had a positive signal response to the presence of streptavidin, and no amplification when both proximity probes were present in solution. The BR5 set had much greater positive amplification (FIG. 2a, Lane 4), indicating that they were good sequence options for single-step, one-pot proximity detection. The bridge-reporter duplexes were also purified using magnetic beads to remove any excess unbound bridge sequences.

### PINATA for Human IL-6 Detection Using Antibody-Oligonucleotide Conjugates in Native PAGE:

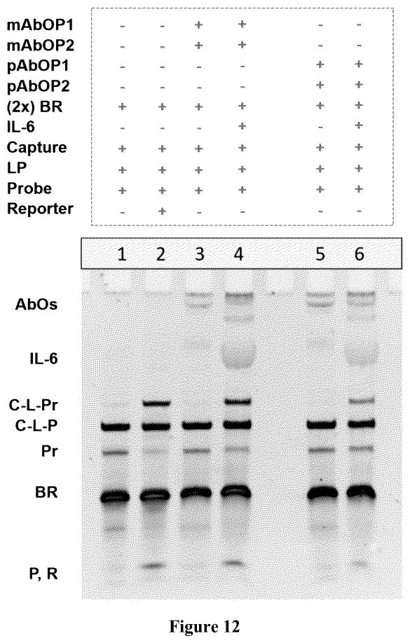

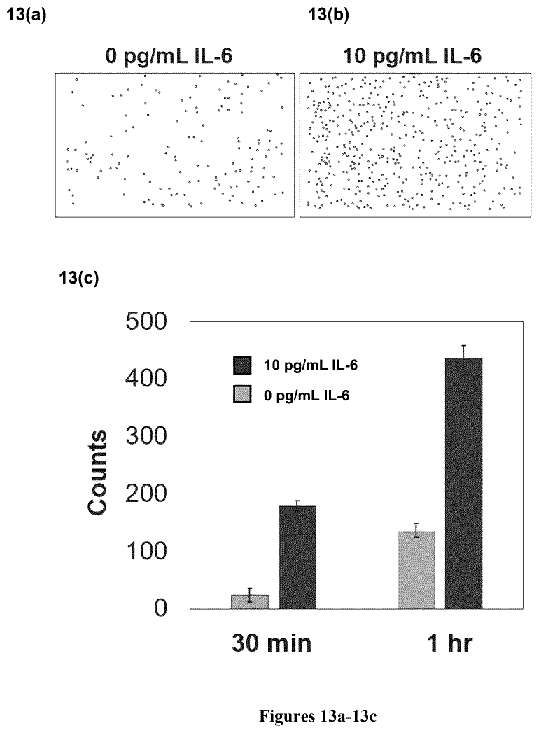

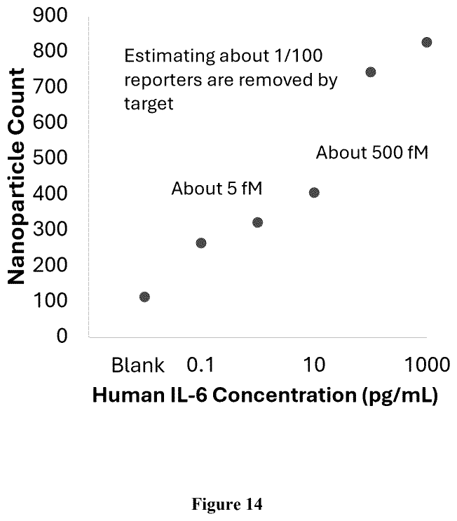

For further testing, the adapter sequences were conjugated to monoclonal mouse, monoclonal rabbit, and polyclonal goat human IL-6 antibodies using antibody-oligonucleotide (AbO) conjugation kits. The AbO conjugates were then hybridized to the proximity probes (monoclonal mouse to proximity probe 1, monoclonal rabbit to proximity probe 2, and the polyclonal goat AbOs were hybridized to either P1 or P2.) Monoclonal and polyclonal AbOs were compared for human IL-6 detection using the BR5 and BR6 probe sets through Native PAGE, along with added nucleic acid target recycling probes (FIG. 12). Lanes 1-3 used monoclonal AbO proximity probes (mAbOP1 and mAbOP2), while Lanes 4-5 used polyclonal AbOs. The monoclonal AbO proximity probe set demonstrated high signal amplification for BR5 through the presence of the final bound nanoparticle complex (capture-linker-NP probe) when human IL-6 was present, even when tested at a tenfold and hundredfold lower concentration than the initial streptavidin optimization (Lanes 2 and 3) in FIG. 12. There was no background amplification in the negative (Lane 1). However, the polyclonal AbOs had a lower signal to noise ratio, with some triplex formation in the negative control (Lane 4) and lower positive signal (Lane 5) in Figure. BR6 showed comparable results with lower positive amplification, as fewer reporters were released.

Feasibility testing for IL-6 detection initially showed some potential non-specific amplification, so the AbO conjugates were purified using magnetic beads to remove any of the excess unbound proximity probes to minimize potential reactions with the bridge-reporter duplex that could trigger amplification without target present. The purified conjugates were tested using native PAGE for both monoclonal and polyclonal antibody-oligonucleotide conjugates. Further optimization using PINATA with PRAM was continued with the purified monoclonal antibody-oligonucleotide conjugates.

PINATA Assay for IL-6 with Digital Detection Using Photonic Resonator Absorption Microscopy:

The PINATA assay was adapted for digital detection of IL-6 using nanoparticle labels on photonic crystals, where the nanoparticle probe sequence was conjugated to gold 80 nm nanoUrchins (AuNPs) that strongly absorb red light (FIG. 2). The capture sequence was immobilized on red-reflecting photonic crystals (FIG. 1) using silanization.

The disclosure herein focuses on digital detection of proteins via enzyme-free ultrasensitive method of protein detection using entropy-driven toehold-mediated strand displacement reactions. The PINATA method utilizes strand displacement nucleic acid amplification, allowing for the use of the same unstable AND circuit approach used to release the reporter in PINATA. The method provides a technical advantage as it can be used with various other standard DNA amplification techniques, both enzymatic and non-enzymatic as well as various other reporting systems and, digitally and non-digitally detected labels. PINATA provides the novel technical effect of applicability with a variety of affinity groups for the detection of different targets and to study protein-protein interactions and kinetics, and detection of various posttranslational modifications in a point-of-care setting. The PINATA assay is a technical advancement over currently available techniques in that a single DNA sequence-selective proximity probe paired with an affinity probe, allows for examination protein-RNA or protein-DNA interactions. The assay provides for ultrasensitive detection of circulating tumor cells or tissue specific exosomes. The use of two DNA sequence-specific proximity probes, allows for detection of nucleic acid targets such as messenger RNA splice variants, circular RNAs, or modified RNA sequences. These targets have been challenging to detect with traditional nucleic acid amplification techniques. PINATA is further advantageous as it has the technical advancement of room temperature assay conditions, linearly amplified signal, and the sensitivity of digital detection.

### REFERENCES

1. Ho, J. E. et al. Protein Biomarkers of Cardiovascular Disease and Mortality in the Community. J Am Heart Assoc 7 (2018).
2. Cho, S.-Y. & Choi, J.-H. Biomarkers of Sepsis. Infect Chemother 46, 1-12 (2014).
3. Hier, D. B. et al. Blood biomarkers for mild traumatic brain injury: a selective review of unresolved issues. Biomarker Research 9, 70 (2021).
4. Borrebaeck, C. A. K. Precision diagnostics: moving towards protein biomarker signatures of clinical utility in cancer. Nature Reviews Cancer 17, 199-204 (2017).
5. Huang, A. T. et al. A systematic review of antibody mediated immunity to coronaviruses: kinetics, correlates of protection, and association with severity. Nature Communications 11, 4704 (2020).
6. Jauset-Rubio, M., Ortiz, M. & O'Sullivan, C. K. Exploiting the Nucleic Acid Nature of Aptamers for Signal Amplification. Biosensors 12, 972 (2022).
7. Jin, B. K., Odongo, S., Radwanska, M. & Magez, S. NANOBODIES®: A Review of Diagnostic and Therapeutic Applications. Int J Mol Sci 24 (2023).
8. Sfragano, P. S., Moro, G., Polo, F. & Palchetti, I. The Role of Peptides in the Design of Electrochemical Biosensors for Clinical Diagnostics. Biosensors 11, 246 (2021).
9. Khodakov, D., Wang, C. & Zhang, D. Y. Diagnostics based on nucleic acid sequence variant profiling: PCR, hybridization, and NGS approaches. Advanced Drug Delivery Reviews 105, 3-19 (2016).
10. Garg, N., Ahmad, F. J. & Kar, S. Recent advances in loop-mediated isothermal amplification (LAMP) for rapid and efficient detection of pathogens. Curr Res Microb Sci 3, 100120 (2022).
11. Murakami, T., Sumaoka, J. & Komiyama, M. Sensitive isothermal detection of nucleic-acid sequence by primer generation-rolling circle amplification. Nucleic Acids Research 37, e19-e19 (2008).
12. Duffy, D. C. Digital detection of proteins. Lab on a Chip 23, 818-847 (2023).
13. Rissin, D. M. et al. Single-molecule enzyme-linked immunosorbent assay detects serum proteins at subfemtomolar concentrations. Nat Biotechnol 28, 595-599 (2010).
14. Huang, Q. et al. Critical Review: digital resolution biomolecular sensing for diagnostics and life science research. Lab on a Chip 20, 2816-2840 (2020).
15. Niemeyer, C. M., Adler, M. & Wacker, R. Detecting antigens by quantitative immuno-PCR. Nat Protoc 2, 1918-1930 (2007).
16. Zhang, J. et al. Real-time immuno-polymerase chain reaction in a 384-well format: Detection of vascular endothelial growth factor and epidermal growth factor-like domain 7. Analytical Biochemistry 463, 61-66 (2014).
17. Chang, L., Li, J. & Wang, L. Immuno-PCR: An ultrasensitive immunoassay for biomolecular detection. Anal Chim Acta 910, 12-24 (2016).
18. Fredriksson, S. et al. Protein detection using proximity-dependent DNA ligation assays. Nat Biotechnol 20, 473-477 (2002).
19. Lundberg, M., Eriksson, A., Tran, B., Assarsson, E. & Fredriksson, S. Homogeneous antibody-based proximity extension assays provide sensitive and specific detection of low-abundant proteins in human blood. Nucleic Acids Res 39, e102 (2011).
20. Greenwood, C. et al. Proximity assays for sensitive quantification of proteins. Biomol Detect Quantif 4, 10-16 (2015).
21. Alam, M. S. Proximity Ligation Assay (PLA). Curr Protoc Immunol 123, e58 (2018).
22. Gullberg, M. et al. A sense of closeness: protein detection by proximity ligation. Current Opinion in Biotechnology 14, 82-86 (2003).
23. Gullberg, M. et al. Cytokine detection by antibody-based proximity ligation. Proc Natl Acad Sci USA 101, 8420-8424 (2004).
24. Löf, L. et al. Detection of Extracellular Vesicles Using Proximity Ligation Assay with Flow Cytometry Readout-ExoPLA. Curr Protoc Cytom 81, 4.8.1-4.8.10 (2017).
25. Liu, R., He, L., Hu, Y., Luo, Z. & Zhang, J. A serological aptamer-assisted proximity ligation assay for COVID-19 diagnosis and seeking neutralizing aptamers. Chemical Science 11, 12157-12164 (2020).
26. Hegazy, M. et al. Proximity Ligation Assay for Detecting Protein-Protein Interactions and Protein Modifications in Cells and Tissues in Situ. Curr Protoc Cell Biol 89, e115 (2020).
27. Lundberg, M. et al. Multiplexed homogeneous proximity ligation assays for high-throughput protein biomarker research in serological material. Mol Cell Proteomics 10, M110.004978 (2011).
28. Wik, L. et al. Proximity Extension Assay in Combination with Next-Generation Sequencing for High-throughput Proteome-wide Analysis. Mol Cell Proteomics 20, 100168 (2021).
29. Petrera, A. et al. Multiplatform Approach for Plasma Proteomics: Complementarity of Olink Proximity Extension Assay Technology to Mass Spectrometry-Based Protein Profiling. J Proteome Res 20, 751-762 (2021).
30. Thorsen, S. B. et al. Detection of serological biomarkers by proximity extension assay for detection of colorectal neoplasias in symptomatic individuals. J Transl Med 11, 253 (2013).
31. Akel, S., Banote, R. K., Asztely, F. & Zelano, J. Protein profiling in plasma for biomarkers of seizure. Epilepsy Res 197, 107241 (2023).
32. Angerfors, A. et al. Proteomic profiling identifies novel inflammation-related plasma proteins associated with ischemic stroke outcome. Journal of Neuroinflammation 20, 224 (2023).
33. Arioz, B. I., Cotuk, A., Yaka, E. C. & Gene, S. Proximity extension assay-based proteomics studies in neurodegenerative disorders and multiple sclerosis. Eur J Neurosci 59, 1348-1358 (2024).
34. Zhang, P. et al. Highly Sensitive Serum Protein Analysis Using Magnetic Bead-Based Proximity Extension Assay. Anal Chem 94, 12481-12489 (2022).
35. Al-Amin, R. A. et al. Sensitive Protein Detection Using Site-Specifically Oligonucleotide-Conjugated Nanobodies. Anal Chem 94, 10054-10061 (2022).
36. Feng, W. et al. NULISA: a proteomic liquid biopsy platform with attomolar sensitivity and high multiplexing. Nature Communications 14, 7238 (2023).
37. Park, Y. S., Choi, S., Jang, H. J. & Yoo, T. H. Assay methods based on proximity-enhanced reactions for detecting non-nucleic acid molecules. Front Bioeng Biotechnol 11, 1188313 (2023).
38. Koos, B. et al. Proximity-dependent initiation of hybridization chain reaction. Nature Communications 6, 7294 (2015).
39. Raykova, D. et al. A method for Boolean analysis of protein interactions at a molecular level. Nature Communications 13, 4755 (2022).
40. Koos, B. & Söderberg, O. Designing and Applying Proximity-Dependent Hybridization Chain Reaction. Curr Protoc Protein Sci 85, 19.28.11-19.28.13 (2016).
41. Wang, X., Gao, H., Qi, H., Gao, Q. & Zhang, C. Proximity Hybridization-Regulated Immunoassay for Cell Surface Protein and Protein-Overexpressing Cancer Cells via Electrochemiluminescence. Anal Chem 90, 3013-3018 (2018).
42. Zhou, H. et al. Proximity binding induced nucleic acid cascade amplification strategy for ultrasensitive homogeneous detection of PSA. Anal Chim Acta 1186, 339123 (2021).
43. Li, J., Zhou, W., Yuan, R. & Xiang, Y. Aptamer proximity recognition-dependent strand translocation for enzyme-free and amplified fluorescent detection of thrombin via catalytic hairpin assembly. Anal Chim Acta 1038, 126-131 (2018).
44. Ren, K., Wu, J., Yan, F. & Ju, H. Ratiometric electrochemical proximity assay for sensitive one-step protein detection. Scientific Reports 4, 4360 (2014).
45. Mocenigo, M. et al. Rapid, Cost-Effective Peptide/Nucleic Acid-Based Platform for Therapeutic Antibody Monitoring in Clinical Samples. ACS Sens 5, 3109-3115 (2020).
46. Porchetta, A. et al. Programmable Nucleic Acid Nanoswitches for the Rapid, Single-Step Detection of Antibodies in Bodily Fluids. J Am Chem Soc 140, 947-953 (2018).
47. Liu, J. N., Huang, Q., Liu, K. K., Singamaneni, S. & Cunningham, B. T. Nanoantenna-Microcavity Hybrids with Highly Cooperative Plasmonic-Photonic Coupling. Nano Lett 17, 7569-7577 (2017).
48. Canady, T. D. et al. Digital-resolution detection of microRNA with single-base selectivity by photonic resonator absorption microscopy. Proceedings of the National Academy of Sciences 116, 19362-19367 (2019).
49. Zhou, L. et al. Bioinspired designer DNA NanoGripper for virus sensing and potential inhibition. Science Robotics 9, eadi2084 (2024).
50. Che, C. et al. Activate capture and digital counting (AC+DC) assay for protein biomarker detection integrated with a self-powered microfluidic cartridge. Lab on a Chip 19, 3943-3953 (2019).
51. Zhao, B., Che, C., Wang, W., Li, N. & Cunningham, B. T. Single-step, wash-free digital immunoassay for rapid quantitative analysis of serological antibody against SARS-CoV-2 by photonic resonator absorption microscopy. Talanta 225, 122004 (2021).
52. Barya, P. et al. Photonic-Plasmonic Coupling Enhanced Fluorescence Enabling Digital-Resolution Ultrasensitive Protein Detection. Small 19, 2207239 (2023).
53. Wang, X. et al. A Target Recycling Amplification Process for the Digital Detection of Exosomal MicroRNAs through Photonic Resonator Absorption Microscopy. Angewandte Chemie International Edition 62, e202217932 (2023).
54. Cunningham, B. Ultrasensitive and ultra selective liquid biopsy: nanophotonics-driven digital resolution biomolecule sensing combined with target molecule recycling. Vol. 12373 PWB (SPIE, 2023).
55. Ghosh, S. et al. A compact photonic resonator absorption microscope for point of care digital resolution nucleic acid molecular diagnostics. Biomed. Opt. Express 12, 4637-4650 (2021).
56. Khemtonglang, K. et al. Portable, smartphone-linked, and miniaturized photonic resonator absorption microscope (PRAM Mini) for point-of-care diagnostics. Biomed. Opt. Express 15, 5691-5705 (2024).
57. Liu, W. et al. Dynamic and large field of view photonic resonator absorption microscopy for ultrasensitive digital resolution detection of nucleic acid and protein biomarkers. Biosensors and Bioelectronics 264, 116643 (2024).
58. Song, T. et al. Fast and compact DNA logic circuits based on single-stranded gates using strand-displacing polymerase. Nature Nanotechnology 14, 1075-1081 (2019).
59. Emanuelson, C., Bardhan, A. & Deiters, A. DNA Logic Gates for Small Molecule Activation Circuits in Cells. ACS Synthetic Biology 13, 538-545 (2024).
60. Jingjing, M. A. Three-input logic gate based on DNA strand displacement reaction. Scientific Reports 13, 15210 (2023).
61. Yurke, B., Turberfield, A. J., Mills, A. P., Simmel, F. C. & Neumann, J. L. A DNA-fuelled molecular machine made of DNA. Nature 406, 605-608 (2000).
62. Zhang, D. Y. & Seelig, G. Dynamic DNA nanotechnology using strand-displacement reactions. Nat Chem 3, 103-113 (2011).
63. Seymour, E., Ünlü, N. L., Carter, E. P., Connor, J. H. & Ünlü, M. S. Configurable Digital Virus Counter on Robust Universal DNA Chips. ACS Sens 6, 229-237 (2021).

1. A system for quantifying proteins in a sample, comprising
a bridge sequence;
a reporter sequence;
two proximity probes;
a solution;
one or more adapters each tethered to an antibody;
a gold nanoparticle (AuNP) probe;
a photonic crystal
a sample; and
an imaging platform,
wherein:
the bridge sequence and reporter sequence are annealed to form a stable bridge-reporter duplex with a exposed nucleotide toehold region at each end of the bridge sequence;
the proximity probes react with the toehold regions;
simultaneous toehold mediated displacement occurs between the proximity probes and the bridge sequence in the presence of a target protein thereby releasing the reporter sequence;
the reporter sequence activates a target recycling amplification process (TRAP) through strand displacement reactions thereby tethering an AuNP probe; and
the imaging platform is configured to visualize and quantify the tethered AuNP probes using photonic resonator adsorption microscopy (PRAM).

2. The system of claim 1 wherein the sample contains the target protein.

3. The system of claim 1, wherein the sample does not contain the target protein.

4. The system of claim 1, wherein the toehold region is comprised of at least six exposed nucleotides.

5. The system of claim 1, wherein the proximity probes are created through antibody oligonucleotide conjugation to form DNA reactive affinity targets.

6. The system of claim 5, wherein the DNA reactive affinity targets can react with the toehold regions.

7. The system of claim 1, wherein the bridge-reporter duplex is stable in the presence of one or both proximity probes and the reporter sequence is stable.

8. The system of claim 1, wherein the presence of a target protein causes simultaneous toehold-mediated displacement reactions between the proximity probe and the bridge sequence thereby releasing the reporter sequence.

9. The system of claim 8, wherein the released reporter sequence is a single stranded DNA (ssDNA).

10. The system of claim 1, wherein the released reporter sequence activates the TRAP.

11. The system of claim 10, wherein TRAP allows for a single reporter to bind multiple AuNP to the photonic crystal for quantification.

12. The system of claim 1, wherein the system can be used to identify post-translational modifications or protein-protein interactions.

13. A biologic assay comprising:
a bridge sequence;
a reporter sequence;
two proximity probes;
a solution; and
one or more adapters each tethered to an antibody; and
a sample;
wherein:
the bridge sequence and reporter sequence are annealed to form a stable bridge-reporter duplex with an exposed toehold region at each end of the bridge sequence;
the proximity probes bound to an antibody-linked adapter can bind a target antigen.

14-15. (canceled)

16. The biological assay of claim 13, wherein the stable duplex has an exposed toehold region on each end comprising of at least six exposed nucleotides.

17. (canceled)

18. The biological assay of claim 13, wherein the proximity probes are created through antibody oligonucleotide conjugation to form DNA reactive affinity targets.

19. The biological assay of claim 18, wherein DNA reactive affinity targets can react with the toehold regions.

20. The biological assay of claim 13, wherein the bridge-reporter duplex is stable in the presence of one or both proximity probes and the reporter sequence is stable.

21. The biological assay of claim 13, wherein the presence of a target protein causes simultaneous toehold-mediated displacement reactions between the proximity probe and the bridge sequence thereby releasing the reporter sequence.

22. The biological assay of claim 21, wherein the released reporter sequence is a single stranded DNA (ssDNA).

23. The biological assay of claim 13, wherein target recycling amplification process (TRAP) and photonic resonator adsorption microscopy (PRAM) are used to quantify protein expression via quantification of gold nanoparticles (AuNP).
